(19) United States
(12) Patent Application Publication
(10) Pub. No.: US 2001/0000176 A1
(43) Pub. Date: Apr. 5, 2001

(54) Method of vaccinating infants against infections

(76) Inventor: Hildegund C. J. Ertl, Villanova, PA (US)

Correspondence Address: Mary E. Bak, Esquire, Howson and Howson, Spring House Corporate Center, Box 457, Spring House, PA, 19477, US

(21) Appl. No.: 09/727,214

(22) Filed: Nov. 30, 2000

Related U.S. Application Data
(63) Continuation of application No. 09/284,525, filed on Apr. 14, 1999.
Application No. 09/284,525 is a 371 of international application No. PCT/US97/19509, filed on Oct. 23, 1997.
(60) Provisional application No. 60/029,463, filed on Oct. 25, 1996.

(51) Int. Cl. A61K 31/70; A01N 43/04

(52) U.S. Cl. 514/44; 424/199.1

(57) ABSTRACT

A method for overcoming maternal inhibition to a vaccine in a mammalian infant under 1 year of age, provided by administering to the infant in a suitable pharmaceutical carrier, a recombinant polynucleotide sequence (i.e., a recombinant virus or DNA vaccine) comprising a sequence encoding an antigen of a pathogenic organism. The polynucleotide vector (i.e., virus or DNA vaccine) useful in this method does not naturally cause a pathogenic infection in the species of the mammalian infant to which the vaccine is administered.

1. This invention was supported by the National Institutes of Health Grant No. AI 33683-04. The United States government has certain rights in this invention.

### FIELD OF THE INVENTION

2. The present invention relates generally to the field of vaccination against infection with a pathogen, and specifically relates to a method for vaccination of infants which overcomes maternal inhibition.

### BACKGROUND OF THE INVENTION

3. Vaccination is the most efficacious medical intervention to reduce and/or prevent morbidity and mortality of humans as well as animals to infectious diseases. Traditionally, vaccines have been based on protein or carbohydrate antigens presented either in the form of whole attenuated pathogens or inactivated pathogens or structural parts thereof. Childhood vaccinations using such traditional vaccines are generally not initiated in humans or domestic animals until the offspring is past the neonatal stage because the immune system is immature at birth.

4. Such delayed vaccination renders young infants susceptible to infections. For example, the neonatal immune system is unable to respond to certain antigens such as bacterial carbohydrates due to a developmental delay of the appropriate B cell subset [D. E. Mosier et al, J. Infect. Dis., 130:14-19 (1977)]. Some antigens such as alloantigens expressed by splenocytes [R. E. Billingham et al, Nature, 172:603 (1953); J. P. Ridge et al, Science, 271:1723 (1996)] induce tolerance in neonatal mice. In contrast, immunization with other antigens induces in the neonatal immune system a preferential Th2 type immune response which does not necessarily provide protection to pathogens. This latter effect was shown to depend in some systems on the dose of the inoculated antigen [T. Forsthuber et al, Science, 271: 1723 (1996); M. Sarzotti et al, Science, 271:1726 (1996)].

5. Neonates (i.e., mammalian infants under 1 year of age, and preferably under 6 months of age) are partially protected against prevalent infections by maternally transferred immune effector mechanisms. Maternal antibodies that, dependent on the host species and the antibody isotype, cross the placenta and/or are transmitted by the milk of immune mothers to the offspring, have multiple effects on the immune status of the offspring. Maternal antibodies protect newborns during the first months of life against infections by numerous viruses [Sheridan et al, Infect. Dis., 149:434-443 (1984); Kohl et al, J. Infect. Dis., 149:38-42 (1984); Reumann et al, J. Immunol., 130:932 (1983)] or bacteria [Lifely et al, Vaccine, 7:17-21 (1989)].

6. However, for other infectious diseases, such as measles virus infection [P. Albrecht et al, J. Ped., 91:715-719 (1977)] or respiratory syncytial virus (RSV) infection [H. W. Kim et al, Am. J. Epidemiol., 98:216-225 (1973)], passively transmitted antibodies are insufficient to protect, and the most severe infections occur in infants under the age of 6 months.

7. Maternally transferred antibodies also can interfere with the development of an immune response upon active immunization of offspring, providing a further impetus to delay childhood vaccinations. Not only can the B cell response be affected, but maternal antibodies as well as syngeneic monoclonal antibodies transferred within 24 hours after birth also inhibit the generation of cytolytic T cells [C. R. M. Bangham, Immunol., 59:37-41 (1986)] and T helper cells [Z. Q. Xiang et al, Virus Res., 24:297 (1992) (Xiang I)].

8. For example, such interference was observed with an experimental malaria vaccine in mice [P. G. Harte et al, J. Clin. Exp. Med., 49:509-516 (1982) (Harte I)], a vaccine against foot-and-mouth disease virus in livestock [M. J. Francis et al, Res. Vet, Sci., 41:33-39 (1986)], a vaccine against measles in human infants [Kim et al, cited above], a rabies vaccine in canines [H. Aghomo, et al, Vet. Res. Corn, 14:415-425 (1977)]; and in mice [Xiang I].

9. Maternal immunity interferes with active immunization well beyond the time span during which the offspring is protected against infection by maternal antibodies, thus rendering them highly susceptible to fatal infections. For example, canine pups from rabies virus-immune bitches have poor antibody responses to a rabies vaccine given before the age of 10 weeks, compared to pups from non-immune bitches. When the prevaccination sera of these pups were tested for residual maternal antibodies to rabies virus, neither antibodies to the G protein nor antibodies to internal proteins could be detected [Aghomo, cited above], thus suggesting that these pups were no longer protected by maternal antibodies at the time of vaccination, which is generally not given to dogs before they are at least 3 months old. A sizable number of human rabies cases are caused by bites from rabid canine pups that are still too young to be eligible for vaccination.

10. The present inventors have shown previously that pups from rabies virus immune dams developed an impaired immune response upon immunization with a traditional rabies virus vaccine, i.e., inactivated rabies virus, resulting in vaccine failures upon subsequent challenge. The degree of vaccine failures was correlated with the amount of maternally transferred antibodies and the age of pups at the time of vaccination [Z. Q. Xiang et al, Virus Res., 24:297 (1992) (xiang I)]. This interference, which affects all aspects of the antigen specific immune response, i.e., B cells, T helper cells [Xiang I], and cytolytic T cells [C. R. M. Bangham, cited above] is thought to be mediated by several mechanisms. Such mechanisms include neutralization of the vaccine by antibodies, tolerization of naive B cells by binding of complexes formed between maternally transferred antibodies and the vaccine, as well as by a putative ‘suppressive’ mechanism induced in the pups by the maternally transferred immune effectors [P. G. Harte et al, J. Clin. Exp. Med., 51:157-164 (1983) (Harte II)].

11. The impairment of the offspring's immune response to active immunization is antigen specific and transient. Nevertheless, although the offspring is protected by maternally transferred immunity for some time during the postnatal period, their inability to mount an efficacious immune response to active immunization can exceed the time span during which maternally transferred immunity provides reliable protection against infection [Xiang I], thus making them susceptible to infections.

12. There remains a need in the art for novel methods of vaccination and novel types of vaccines, which induce a protective immune response in neonates and young humans and animals in the presence of maternally transferred immune mechanisms.

### SUMMARY OF THE INVENTION

13. The invention provides methods for overcoming maternal inhibition to a vaccine or therapeutic compositions in a mammalian infant or neonate.

14. In one aspect, the method comprises administering at a suitable dose to the infant/neonate in a suitable pharmaceutical carrier, a transcribable polynucleotide sequence comprising a sequence encoding an antigen of a pathogenic organism. The polynucleotide sequence may be a recombinant vector, such as a replication-defective virus or a plasmid vector, also called a DNA vaccine. In one embodiment the polynucleotide sequence is a recombinant virus vector, which may be adenovirus, canarypox virus, retrovirus, etc., that does not naturally infect the species of the mammalian infant. In a particularly desirable embodiment, the neonate is human and the virus is an adenovirus of non-human, i.e., bovine or other species, origin.

15. In another aspect, the invention provides the use of such transcribable polynucleotide sequences, i.e., recombinant replication-defective viruses and DNA vaccines, in the preparation of medicaments useful in the methods described above.

16. In still a further aspect, the method described above is a veterinary method and the infant is an animal such as a domestic pet or livestock. Preferably, the vector carrying the polynucleotide sequence does not naturally infect the species of the mammalian infant, although for veterinary uses, a human pathogen may be used.

17. Other aspects and advantages of the present invention are described further in the following detailed description of the preferred embodiments thereof.

### BRIEF DESCRIPTION OF THE DRAWINGS

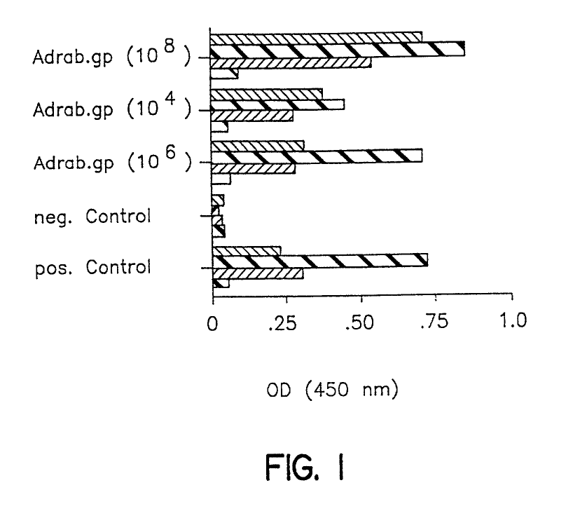

18. FIG. 1 shows the isotype profiles of the B cell response upon neonatal immunization with a recombinant adenovirus vector carrying the rabies glycoprotein gene (Adrab.gp) (see Examples 1C4 and 3C). Data present the mean of duplicates. Standard errors were for each data point below 10% of the mean. The symbols are: box with thin back slash, IgG1; box with heavy backslash, IgG2a; box with thin slash, IgG2b; box with heavy slash, IgG3.

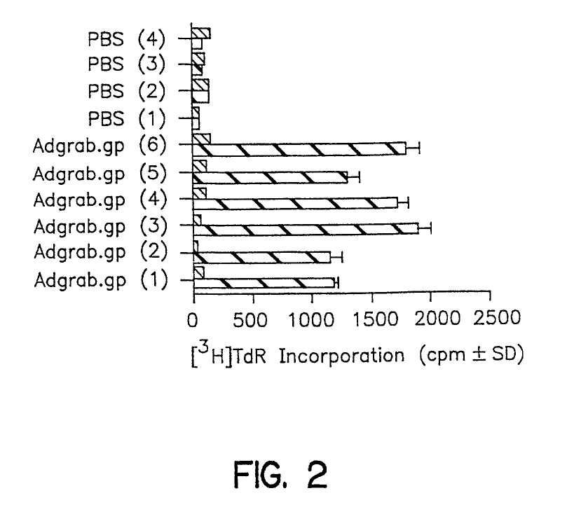

19. FIG. 2 is a graph of cytokine release by splenocytes of mice immunized as neonates with Adrab.gp (see Example 3D). The symbols are box with thin back slash, representing splenocytes from mice cultured in medium and box with heavy backslash, representing splenocytes co-cultured with an inactivated rabies virus, ERA-BPL (See Example 1C1). Data are expressed as the mean of triplicates ±standard deviations of the mean and the graph plots vaccine vs. proliferation of the HT-2 indicator cell line.

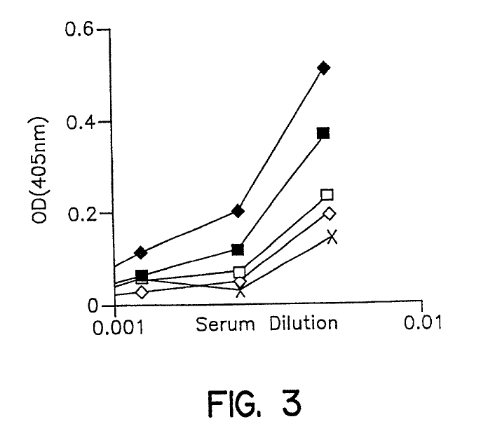

20. FIG. 3 is a graph of the antibody response measured in optical density at 405 nm wavelength (OD₄₀₅) to a “genetic vaccine”, pSG5rab.gp, or the inactivated virus, ERA-BPL, in the sera of young adult mice born to naive or rabies virus immune dams. pSG5rab.gp is a plasmid vector which expresses the rabies virus glycoprotein under the control of the SV-40 gene (See Example 1D). Groups of C3H/He mice born to naive (filled symbols) or rabies virus immune (open symbols) dams were vaccinated at 6 weeks of age with 10 μg of ERA-BPL virus (diamonds) or 50 μg of pSG5rab.gp vector (squares). Mice were bled 6 weeks later and serum antibody titers to rabies virus were determined by an enzyme linked immunosorbent assay (ELISA) using sera from age-matched naive mice (X) for comparison. Number of pups per groups: 13 pups from ERA-dams were immunized with ERA; 3 pups from naive dams were immunized with ERA; 11 pups from ERA dams were immunized with pSG5rab.gp; and 9 pups from naive dams were immunized with pSG5rab.gp.

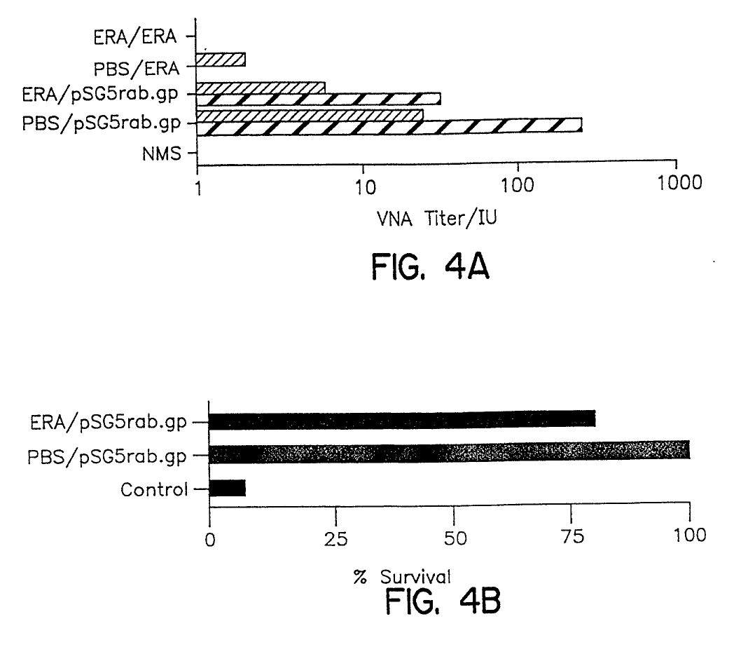

21. FIG. 4A is a bar graph showing the induction of virus neutralizing antigens (VNA) and protection to challenge upon vaccination of young adult mice. The same sera tested in FIG. 3 by an ELISA were tested for VNA to rabies virus using a standardized NIH reference serum for comparison. Ten international units (IU) is the equivalent of a VNA titer of 1:13 5. In addition, some of the groups of mice were challenged with live rabies virus (see FIG. 4B) and sera of surviving animals were harvested 4 weeks later and tested in parallel with the pre-challenge sera. The Y axis refers to the following groups: ERA/ERA: mice born to rabies virus-immune dams vaccinated with the inactivated rabies virus vaccine; PBS/ERA: mice born to naive, i.e., phosphate buffered saline (PBS) inoculated, dams vaccinated with the inactivated rabies virus vaccine; ERA/pSG5rab.gp: mice born to ERA immune dams vaccinated with the genetic vaccine; PBS/pSG5rab.gp: mice from naive dams immunized with pSG5rab.gp; and NMS: sera harvested from age matched control mice. The symbols are: prechallenge (light cross-hatched square); post-challenge (dark cross-hatched square). Data are expressed as IU calculated in comparison to the reference serum. VNA titers in the ERA/ERA and NMS groups were below the level of detectability.

22. FIG. 4B is a graph illustrating percent survival of pSG5rab.gp vaccinated mice as well as the control mice from the same groups identified in FIG. 4A, that were challenged with 10 LD₅₀ (i.e., ten times the dose which is lethal to 50% of the challenged animals) of live infectious rabies CVS-24 virus. Survival was recorded over a 4 week observation period.

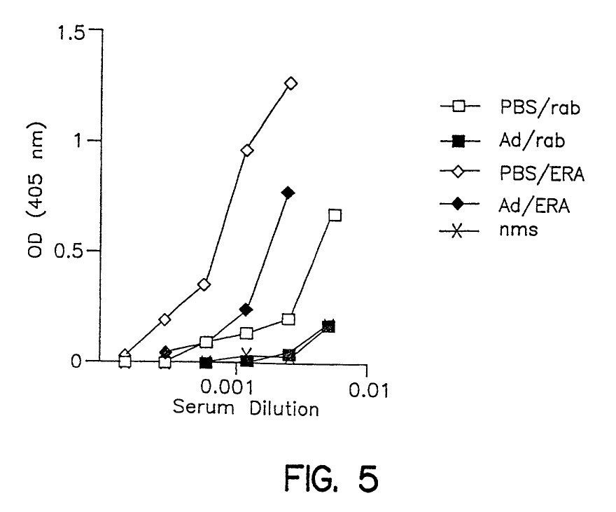

23. FIG. 5 is a graph showing the effect of maternal immunization with a recombinant adenovirus vaccine expressing the rabies virus glycoprotein on the B cell response to genetic immunization. Female C3H/He mice were immunized twice prior to mating with either 10⁶ pfu of Adrab.gp virus or with PBS. Pups from the Adrab.gp vaccinated dams were themselves vaccinated at 6 weeks of age with 5 μg of ERA-BPL virus (Ad/ERA, closed diamond) or with 50 μg of pSG5rab.gp vector (Ad/rab, closed square). Pups from the PBS vaccinated dams were themselves vaccinated at 6 weeks of age with 5 μg of ERA-BPL virus (PBS/ERA, open diamond) or with 50 μg of pSG5rab.gp vector (PBS/rab, open square) were bled 6 weeks later and serum antibody titers were determined by an ELISA using age-matched normal mouse sera (nms, X) for comparison.

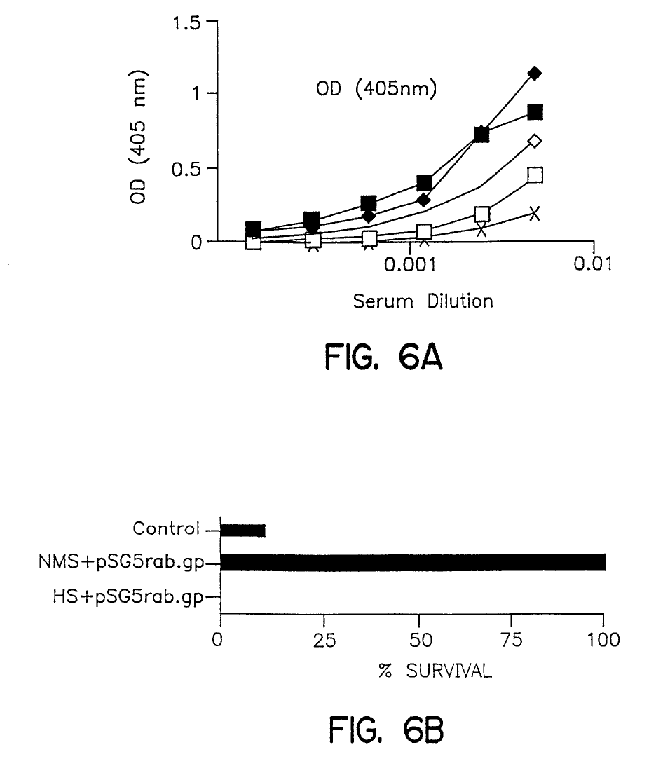

24. FIG. 6A is a graph illustrating the effect of passive transfer of antibodies to rabies virus on the antibody response of young adult mice. One group of mice were inoculated with 10 IU of a hyperimmune serum to rabies virus, resulting in a serum antibody titer of 3 IU measured 24 hours later. Control mice received an equivalent dose of a control serum preparation. Four days later, both groups of mice were vaccinated with either 10 μg of ERA-BPL virus or 50 μg of pSG5rab.gp vector. Antibody titers were determined by an ELISA 6 weeks later using a normal mouse serum for comparison. Data is recorded as OD₄₀₅ vs. serum dilution. The symbols are: mice receiving the hyperimmune serum followed by the ERA-BPL virus (open diamond); mice receiving the hyperimmune serum followed by the pSG5rab.gp vector (open square); control mice receiving the ERA-BPL virus (closed diamond); control mice receiving the vector (closed square); normal mouse serum (X).

25. FIG. 6B is a bar graph illustrating the percentage survival of two groups of genetically immunized mice (NMS+pSG5rab.gp represents the mice receiving normal mouse serum and the genetic vaccine; HS+pSG5rab.gp represents the mice receiving hyperimmune serum and the genetic vaccine) and age matched naive mice (Control), which were challenged with 10 LD₅₀ of CVS-24 virus.

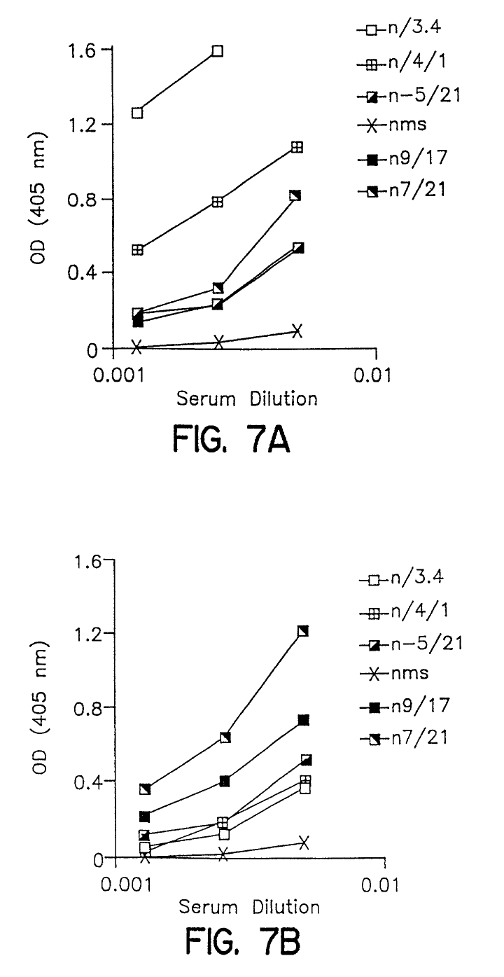

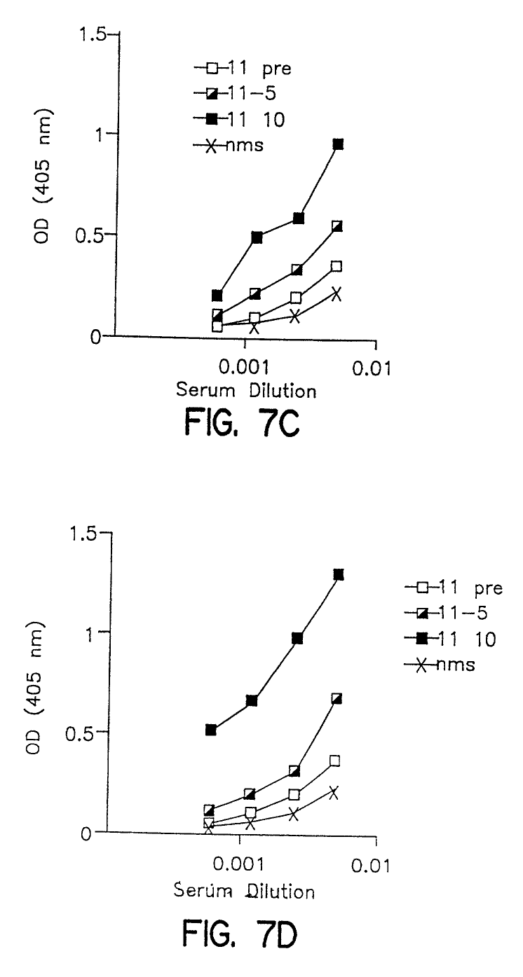

26. FIG. 7A is a graph showing the effect of maternally transferred immunity on the B cell response upon genetic immunization of neonates. Pups born to rabies virus immune (ERA-BPL vaccinated) dams were inoculated within 48 hours after birth with pSG5rab.gp vector. Mice were bled 1 month ▭, 2 months (cross-hatched square), 4 months (diagonally shaded square with dark shade on lower right diagonal), 6 months (diagonally shaded square with dark shade on upper right diagonal) and 8 (▪) months later, and serum antibody titers were determined by an ELISA using a normal mouse serum from 8-10 week old mice for comparison. The symbol X represents normal mouse serum. Data is plotted as OD₄₀₅ vs. serum dilution.

27. FIG. 7B is a graph reporting similar results for a similar experiment, except that the pups so treated were born to sham-vaccinated (naive) dams. Symbols and results are reported as described in FIG. 7A.

28. FIG. 7C is a graph showing the booster effect on the same group of pups of FIG. 7A, boosted at 10 months of age with an E1-deleted adenoviral recombinant expressing the rabies virus glycoprotein Serum antibody titers are measured before boosting (□) and 5 (diagonally shaded square with dark shade on lower right diagonal) and 10 (▪) days following vaccination with the adenoviral recombinant. The symbol X represents normal mouse serum.

29. FIG. 7D is a graph showing the booster effect on the same group of pups of FIG. 7B, treated as described in FIG. 7C. Symbols and data are as for FIG. 7C.

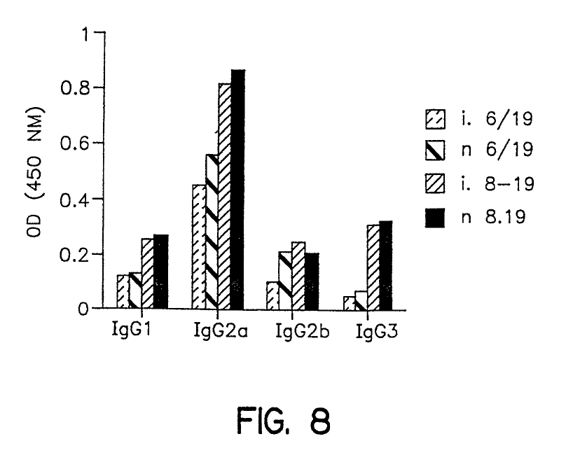

30. FIG. 8 is a bar graph illustrating the effect of maternally transferred immunity on the isotype profile of antibodies to rabies virus induced by the genetic vaccine. Sera of mice immunized at birth with the pSGSrab.gp vector as described in FIG. 6A were tested for the isotype distribution of antibodies to rabies virus. Sera were negative for IgM and IgA (not shown). The bar symbols are: sera of pups from immune dams harvested at 6 (broken cross-hatching) and 8 (thin line cross-hatching) months of age; sera of pups from naive dams harvested at 6 (thick line cross hatching) and 8 (black bar) months of age. Data show the mean of triplicate measurements+SD.

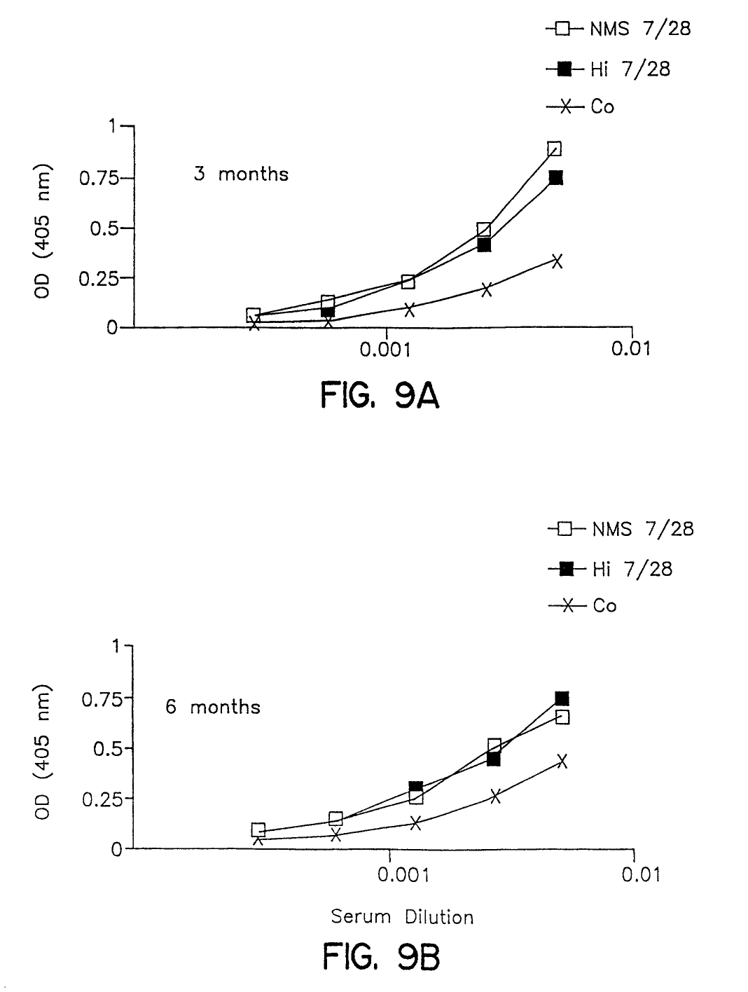

31. FIG. 9A is a graph showing the effect of passive immunization of neonates on the antibody response to a genetic vaccine. Pups born to rabies virus immune or naive dams were inoculated within 24 hours after birth with 10 IU of a hyperimmune serum to rabies virus (closed square) or an equivalent dose of normal mouse serum (open square). Pups were bled 3 months later and serum antibody titers to rabies virus were determined by an ELISA using sera from age-matched naive mice for comparison (X). Data is reported as OD₄₀₅ vs. serum dilution.

32. FIG. 9B is a graph showing the same experiment as described in FIG. 9A, except that the results were measured by bleeding the pups 6 months later. Symbols and data are reported as described in FIG. 9A.

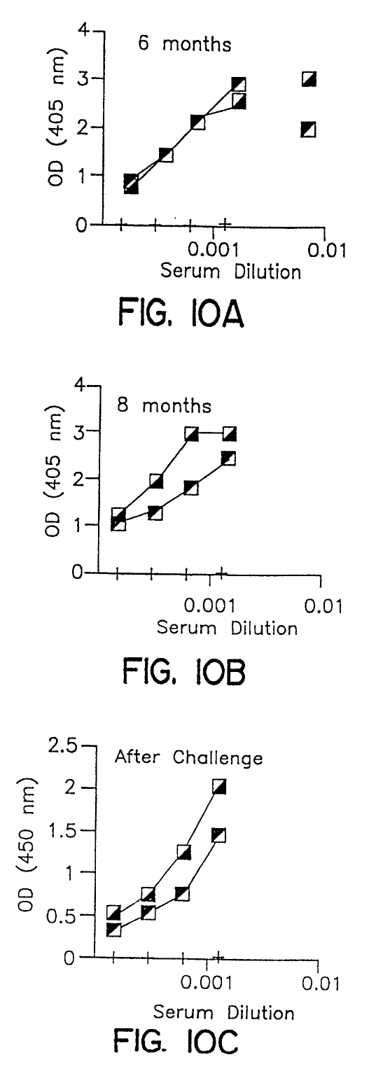

33. FIG. 10A is a graph illustrating the effect of maternally transferred antibodies on the antibody response to neonatal vaccination with Adrab.gp virus. Mice from naive (upper left diagonal filled square) (7 mice) or ERA-BPL immune (lower right diagonal filled square) (10 mice) dams were immunized at birth with about 4×10⁴ pfu of Adrab.gpvirus. Mice were bled at 6 months of age, and antibodies to rabies virus were determined, Sera from age-matched naive mice (3) is indicated by “+”.

34. FIG. 10B is a study performed in parallel with that of FIG. 10A, but the mice were bled at 8 months of age. Symbols are the same as for FIG. 10A.

35. FIG. 10C is a graph of the titers of antibody of the mice of FIGS. 10A and 10B which were subsequently challenged wth CVS-24 virus, bled 21 days after challenge and the titers determined by a separate ELISA. The symbols are the same as for FIG. 10A.

### DETAILED DESCRIPTION OF THE INVENTION

36. The inventors provide herein a method of vaccinating newborn or neonate mammals which overcomes the maternal inhibition which prevents successfull vaccination in prior art methods. The method of the invention involves administering a suitable dose of a transcribable polynucleotide composition comprising a sequence encoding a desired antigen to the neonate.

37. As used herein, the term “mammalian neonate” or “infant” includes newborn mammals having circulating maternal antibodies. For example, where reference is made to humans, a neonate is generally less than 12 months old; for canines, the neonate is generally less than 16 weeks old; for felines, the neonate is generally less than 16 weeks old. However, in general, this method may be employed on all mammalian infants under 1 year of age. Based on this information, the skilled artisan can readily determine the appropriate age range for the selected mammalian neonate vaccinee.

38. As used herein, the term “transcribable polynucleotide composition” includes a recombinant replication-defective virus vaccine or a plasmid vector vaccine which includes a sequence encoding the antigen of choice which may be transcribed into the antigen when administered to the subject. Prefrably, a transcribable polynucleotide encoding the antigen is under the regulatory control of a promoter sequence. These compositions permit expression of the desired antigen-encoding gene and/or gene product. Desirably, these compositions are suitable for administration to the mammalian species to be vaccinated in that they are non-pathogenic in the selected species.

39. As used herein, “suitable dose” refers to the concentration of vector particles (usually in μgs) or recombinant virus vectors (usually in plaque forming units (pfu)) which induces the desired immune response. In one embodiment, such a dose is the lowest useful dose to induce the response. For example, where the composition is an E1-deleted adenovirus containing the rabies glycoprotein gene (e.g., the Adrab.gp viral vector exemplified herein) a suitable dose refers to about 10³ to about 10⁷ plaque forming units (pfu). Where the vaccine composition is a plasmid DNA bearing the rabies glycoprotein gene (e.g., pSG5rab.gp), a suitable dose refers to between about 0.5 to about 5 mg. One of skill in the art can readily select appropriate concentrations of other vaccine vectors for use in the method of the invention.

40. I. Polynucleotide Compositions Useful in the Invention

41. The polynucleotide compositions useful in the method of the invention, as defined above, may be readily selected by one of skill in the art.

42. A. Recombinant Viral Vectors

43. In one embodiment, the polynucleotide composition is a recombinant virus. Preferably the virus is a replication-defective virus. Such viral vectors are well known to those of skill in the art. See, e.g., S. Plotkin et al, European Patent Application No. 389,286, published Sep. 26, 1990; Davis, U.S. Pat. No. 4,920,309; L. Prevac, J. Infect. Dis., 161:27-30 (1990); T. Ragot et al, J. Gen. Virol., 74:501-507 (1993); M. Eliot et al, J. Gen. Virol., 71:2425-2431 (1990); and S. C. Jacobs et al, J. Virol., 66:2086-2095 (1992); and Z. Xiang et al, Virology, 219(1):220-227 (1996) (Xiang III)]. Particularly suitable for use in the method of the invention are recombinant viral vectors derived from adenovirus [see, e.g., U.S. Pat. No. 5,494,807; U.S. Pat. No. 5,494,671; U.S. Pat. No. 5,443,964; and B. Brochier et al, Vaccine, 12:1368-1371 (1994)], pox viruses, [W. Cox et al, Virology, 195(2):845-850 (1993) and J. Tartaglia et al, J. Virol., 67(4):2370-2375 (1993)], and retrovirus [J. Tartaglia et al, AIDS Research and Human Retroviruses, 9(Suppl. 1):S27 (1993)]. Such viral vectors can be readily selected and prepared by the skilled artisan.

44. Desirably, the viral vector selected for use in the method of the invention is derived from a virus which is not pathogenic in the mammalian neonate selected. For example, where the vaccinate is a non-human mammal, e.g., dogs and cats, human adenovirus strains are highly suitable. Similar constructs based on non-human strains of adenovirus may be used in the method of the invention where the vaccinate is a human newborn. See, for example, the bovine adenovirus construct described in International Patent Application No. WO95/16048, published Jun. 15, 1995. Alternatively, one may selected a canarypox or other non-human pathogenic viral vector for use in the method of the invention where the vaccinate is a human neonate.

45. Currently, in one embodiment, the vector used in the method of the invention is an E1 deleted recombinant adenovirus. These vectors are safe due to their relative inability to replicate; they induce a potent immune response even if given shortly after birth and at low doses; and they are only slightly and transiently affected by maternal immunity to the expressed antigen. In a particularly preferred embodiment, the recombinant, E1-deleted recombinant adenovirus expresses the rabies virus glycoprotein. The inventors have shown with the data provided herein that Adrab.gp given subcutaneously to mice during the neonatal period induce an immune response to rabies virus even in the presence of maternally transferred immunity to rabies virus. Most importantly pups from rabies virus immune dams, as well as those from naive dams immunized at birth with a comparatively low dose of the Adrab.gp vaccine, were completely protected to rabies virus given at 9 months of age. The inventors have clearly shown that the Adrab.gp vaccine overcomes inhibition by maternally transferred immunity, even if given to neonatal mice which have high titers of maternal antibodies, i.e., more than about 45-105 units of neutralization titer.

46. B. DNA Vaccines

47. DNA molecules carrying a pathogen's gene under the control of a suitable promoter can readily transfect cells in situ upon inoculation into skin or muscle tissue and cause expression of the encoded protein and, in consequence, induce of a specific B and T cell-mediated immune response. The use of sophisticated propulsion devises or simple syringes to administer such vectors and the consequences thereof have led to the era of “genetic” vaccines, also commonly referred to as DNA vaccines.

48. DNA vaccines are small circular pieces of DNA composed of a backbone for amplification and selection in bacteria and a transcriptional unit for translation of the pathogens' gene in mammalian cells. Such vaccines have a number of advantages over more traditional types of vaccines. One of the main advantages of vector vaccines, at least for experimenters, is the ease with which they can be constructed and manipulated.

49. Immunologically, genetic vaccines provide their own adjuvant in form of CpG sequences present in the bacterial backbone. DNA vaccines cause de novo synthesis of proteins in transfected cells, leading to association of antigenic peptides with MHC class I determinants and hence to activation of cytolytic T cells. In addition DNA vaccines do not elicit measurable immune responses to the carrier (i.e., the vector DNA) thus allowing their repeated use.

50. Thus, in an alternate embodiment of this invention, the polynucleotide composition comprises a DNA sequence encoding the selected antigen without a viral carrier. The DNA sequences, together with nucleotide sequences encoding appropriate promoter sequences, may be employed directly (“naked DNA”) as a therapeutic composition according to this invention [See, e.g., J. Cohen, Science, 259:1691-1692 (Mar. 19, 1993); E. Fynan et al, Proc. Natl. Acad. Sci., 90: 11478-11482 (Dec. 1993); J. A. Wolff et al, Biotechniques, 11:474-485 (1991); International Patent Application PCT W094/01139, published Jan. 20, 1994, which describe similar uses of ‘naked DNA’, all incorporated by reference herein.

51. To prepare a DNA vaccine, briefly, the DNA encoding the antigen of choice may be inserted into a nucleic acid cassette. This cassette may be engineered to contain, in addition to the antigen sequence to be expressed, other optional flanking sequences which enable its association with regulatory sequences. This cassette may then optionally be inserted downstream of a promoter, an mRNA leader sequence, an initiation site and other regulatory sequences capable of directing the replication and expression of the antigen encoding sequence in vivo.

52. Suitable plasmid vaccines may be readily prepared by one skilled in the art. See, e.g., J. Sambrook et al, Molecular Cloning: A Laboratory Manual, 2d ed., Cold Spring Harbor Laboratory Press (1989). One particularly desired plasmid vaccine useful in the prevention of rabies is pSG5rab.gp [Z. Q. Xiang et al, Virology, 199:132-140 (1994) (Xiang II)]. This vaccine can be used to express the selected antigenic or immunogenic protein in vivo. [See. e.g., J. Cohen, Science, 259:1691-1692 (March, 1993); E. Fynan et al, Proc. Natl. Acad. Sci., 90:11478-11482 (Dec. 1993); J. A. Wolff et al, Biotechniques, 11:474-485 (1991)].

53. C. The Sequence Encoding the Antigen

54. Regardless of the type of vector or DNA vaccine composition selected, as described above, one of skill in the art can readily select a nucleic acid, and preferably a DNA sequence encoding an antigen, immunogenic polypeptide, or other desired gene product which is to be engineered into and administered according to the method of this invention. For convenience, reference is made herein to an antigen. However, it will be understood that immunogenic polypeptides or other gene products desirable for administration in a vaccine may be substituted. Such a nucleic acid sequence is desirably heterologous to the vector used for delivery or to the promoter with which the encoding sequence is associated. The selection of the nucleic acid sequences is not a limitation of the present invention.

55. For ease of understanding, the following disclosure describes the selected antigen as a rabies glycoprotein. While the examples herein are limited to the use of a rabies glycoprotein, one of skill in the art will readily understand that any other sequence encoding a pathogenic antigen or fragment thereof may be used in developing vaccine constructs for use in the method of this invention, e.g., by replacing the rabies glycoprotein encoding sequence of the exemplified constructs with other antigen-encoding sequences from other pathogens, including those discussed below.

56. Therefore, some suitable antigens may include, without limitation, a polynucleotide sequence encoding a peptide or protein from rabies virus, human immunodeficiency virus (HIV), respiratory syncytial virus (RSV), rotavirus and measles virus.

57. 1. Rabies Virus Antigens

58. In an exemplary particularly preferred embodiment, the antigen is the rabies glycoprotein [see, U.S. Pat. No. 4,393,201]. A variety of rabies strains are well known and available from academic and commercial sources, including depositaries such as the American Type Culture Collection, or may be isolated using known techniques. The strain used in the examples below is the Evelyn Rockitniki Abelseth (ERA) strain. However, this invention is not limited by the selection of the rabies strain or this particular antigen.

59. 2. HIV Antigens

60. For example, where the condition is human immunodeficiency virus (HIV) infection, the protein is preferably HIV glycoprotein gp120 for which sequences are available from GenBank. Also useful in such vaccines are other HIV proteins or antigens disclosed in the art, such as gp160, gp41, and the tat gene [see, International Patent Application No. W092/14755, published Sep. 3, 1992; see, also, G. Meyers et al., Human retroviruses and AIDS 1993, I-V. A compilation and analysis of nucleic acid and amino acid sequences. Los Alamos National Laboratory, Los Alamos, N.M.].

61. 3. RSV Antigens

62. RSV is pleomorphic and ranges in size from 150-300 nm in diameter. The RNA genome encodes 10 unique viral polypeptides ranging in size from 9.5 kDa to 160 kDa [Huang, Y. T. and G. W. Wertz, J. Virol. 43:150-157 (1982)]. Seven proteins (F, G, N, P, L, M, M2) are present in RSV virions and at least three proteins (F, G, and SH) are expressed on the surface of infected cells. The F protein has been conclusively identified as the protein responsible for cell fusion since specific antibodies to this protein inhibit syncytia formation in vitro and cells infected with vaccinia virus expressing, recombinant F protein form syncytia in the absence of other RSV virus proteins.

63. Where prevention of respiratory syncytial virus infection is desired, the protein is selected from the above-listed antigens, but particularly the surface attachment (G) glycoprotein [Johnson, R. A. et al., Proc. Nat'l. Acad. Sci. USA 84:5625-5629 (1987)] and the fusion (F) protein, for which sequences are available from GenBank. See, also, the epitopes disclosed in International patent publication No. W092/043 81, published Mar. 19, 1992, and International patent publication No. W093/20210, published Oct. 14, 1993. Still other antigen encoding sequences may be selected for this use, as described in McIntosh, K. and R. M. Chanock, In: “Respiratory Syncytial Virus”, Ch. 38, B.N. Fields ed., Raven Press (1990) and Hall, C. B., In: “Textbook of Pediatric Disease” Feigin and Cherry, eds., W. B. Saunders, pgs 1247-1268 (1987).

64. Thus, numerous antigen-encoding sequences may be selected from various strains and serotypes of RSV for use in a vaccine according to this invention.

65. 4. Rotavirus Antigens

66. Rotaviruses have an inner and outer capsid with a double-stranded RNA genome formed by eleven gene segments. Two outer capsid proteins, v.p.7 and v.p.4, are the determinants of virus serotype. The v.p.7 protein is coded for by either gene segment 7, gene segment 8 or gene segment 9 of the particular human rotavirus. For other antigenic sequences, see, for example, U.S. Pat. No. 5,626,851; G. Larralde et al, J. Virol.,65:3213-3218 (1991); U.S. Pat. No. 5,298,244; U.S. Pat. No. 4,190,645, U.S. Pat. No. 5,332,658; V. Gouvea et al, J. Infect. Dis., 162:362-367 (1990), P. Woods et al, J. Clin. Microbiol., 30:781-785 (1992), and J. Gentsch et al, J. Clin. Microbiol., 30:1365-1373 (1992)].

67. Thus, numerous antigen-encoding sequences may be selected from various strains and serotypes of rotavirus for use in a vaccine according to this invention.

68. 5. Other Antigens, including Those From Animal Pathogens

69. In addition to these proteins, other pathogen-associated proteins are readily available to those of skill in the art. A non-inclusive list involves antigen sequences from disease of domestic animals, e.g., canine parvovirus, feline immunodeficiency virus, etc. Similarly antigenic sequences may be selected from pathogens which prey on livestock, horses, or other valuable animals for use in the methods and constructs of this invention.

70. Antigenic sequences from a host of other infectious agents affecting humans, particularly children may also be selected for use in this invention. The sequences encoding these and other suitable antigens may be readily obtained and selected by the skilled artisan for use in preparing a recombinant virus, plasmid vectors or DNA vaccines useful in the method of the invention.

71. II. Formulation of Vaccine

72. A recombinant vector bearing a heterologous nucleic acid sequence encoding an antigen, as described above, may be administered to a human or non-human animal neonate, preferably suspended in a biologically compatible solution or pharmaceutically acceptable delivery vehicle or carrier. A suitable vehicle is water or sterile saline. Other aqueous and non-aqueous isotonic sterile injection solutions and aqueous and non-aqueous sterile suspensions, including balanced salt solutions, and protein solutons, and other solutions known to be pharmaceutically acceptable carriers and well known to those of skill in the art may be employed for this purpose.

73. Optionally, a vaccinal composition of the invention may be formulated to contain other components, including, e.g. adjuvants, stabilizers, pH adjusters, preservatives and the like. Such components are well known to those of skill in the vaccine art.

74. For example, in one desired embodiment, the vaccine composition of the invention further comprises cytokines or co-stimulatory signals. Suitable cytokines and co-stimulatory signals include, without limitation, granulocyte macrophage colony stimulating factor (GM-CSF), interleukin 2, (IL-2), IL-3, IL-4, IL-5, IL-10, IL-12, IL-13, IFN-γ, B7.1, IL-2, IL-12, and the like. Desirably, these cytokines are of the same mammalian origin as the species to which the vaccine composition is being administered.

75. III. Administration of Vaccine

76. The recombinant vectors are administered in an “effective amount”, that is, an amount that is effective in a selected route of administration to transfect or infect the desired cells and provide sufficient levels of expression of the selected gene to provide a vaccinal benefit, i.e., protective immunity.

77. Conventional and pharmaceutically acceptable routes of administration may include intranasal, intramuscular, subcutaneous, intradermal, rectal, vaginal, oral and other parental routes of administration. Routes of administration may be combined, if desired, or adjusted depending upon the vector, the immunogen or the disease. For example, where the vector is canarypox, oral administration may be desired. As another example, in prophylaxis of rabies, the subcutaneous or intramuscular routes are preferred. The route of administration primarily will depend on the nature of the disease being treated prophylactically.

78. Doses or effective amounts of the recombinant vector will be readily determined by the skilled artisan, depending upon the factors such as the selected antigen, the age, weight and health of the animal, and the selected animal species. For example, a prophylactically effective amount or dose of the Adrab.gp vaccine useful in preventing rabies is generally in the range of from about 100 μl to about 10 ml of saline solution containing concentrations of from about 1×10⁴ to 1×10⁷ plaque forming units (pfu) virus/ml. A preferred dose is from about 1 to about 10 ml saline solution at the above concentrations. The levels of immunity of the selected gene can be monitored to determine the need, if any, for boosters.

79. Currently, when vaccinating against rabies, the preferred dose is about 10⁵ pfu of the recombinant virus per mouse, preferably suspended in about 0.1 mL saline. Thus, when vaccinating against rabies infection, a larger animal would preferably be administered about a 1 mL dose containing about 1×10⁶ Adrab.gp pfu suspended in saline. Following an assessment of antibody titers in the serum, optional booster immunizations may be desired.

80. In one desired embodiment, the vaccine composition of the invention may be administered in conjunction with cytokines, as described above. Where not included in the vaccine formulation, these cytokines may be administered separately using suitable techniques. For example, nucleic acid sequences encoding these cytokines may be administered such that the cytokines are expressed in vivo. Alternatively, the cytokines may be formulated into a composition using a suitable carrier or delivery system. Suitable formulations and modes of administration may be readily selected by one skilled in the art.

81. The following examples illustrate various aspects of the present invention. These examples do not limit the scope of the invention, which is embodied in the appended claims.

### EXAMPLE 1

### Experimental Materials and Assays

82. A. Mice

83. Male and female C3H/He mice were purchased from Jackson Laboratories, Bar Harbor, Me. They were bred at The Animal Facility of The Wistar Institute by co-housing 2 females with one male. Mice were separated once pregnancies were established. Pups were separated from their dams according to sex at 4 weeks of age. Mice of both sexes equally distributed between the different groups were used for the experiments.

84. B. Cells

85. Baby hamster kidney (BHK)-21 cells and HeLa cells were maintained in Dulbeccos modified Eagles medium (DMEM) supplemented with glutamine, non essential amino acids, sodium pyruvate, HEPES buffer, antibiotics (culture medium) and 10% heat-inactivated fetal bovine serum (FBS), in a humidified 10% CO₂ incubator. HEK293 cells were maintained in DMEM supplemented with 10% FBS, glutamine and antibiotics in 5% CO₂ humidified incubator. HT-2 cells were maintained in culture medium supplemented with 10% FBS and 10% rat Concanavalin A supernatant as a source of lymphokines, and 10⁻⁶ M 2-mercaptoethanol. The IL-4 dependent CT4S cell line was maintained in culture medium without HEPES buffer supplemented with 10% FBS and 10 units per ml of recombinant Interleukin (IL)-4.

86. C. Viruses

87. 1. ERA-BRL

88. Rabies virus of the Evelyn Rokitniki Abelseth (ERA) strain was grown on BHK-2 1 cells. The ERA virus was purified, inactivated with betapropionolactone (BPL) and adjusted to a protein concentration of 0.1 mg/ml as described in T. J. Wiktor, in “Laboratory Techniques in Rabies”, (M. Kaplan and H. Koprowski, eds.), 2nd ed., Vol 23; 101-120 WHO Monograph, Geneva (1 973) [Wiktor I).

89. 2. CVS-24

90. Rabies virus of the challenge virus strain (CVS)-24 virus was propagated in the brain of suckling ICR virus and titrated in adult C3H/He mice by intramuscular (i.m.) inoculation [T. J. Wiktor et al, J. Virol., 21:626-633 (1977) (Wiktor II)]. To establish the mean lethal dose (LD50), CVS-24 virus was titrated upon intramuscular inoculation (i.m.) of outbred adult ICR mice.

91. 3. CVS-11

92. Rabies virus of the challenge strain (CVS)-11 strain of rabies virus was propagated on BHK-21 cells, and titrated on BHK21 cells to determine the optimal dose for virus neutralization assays.

93. 4. Adrab.gp

94. An E1-deleted replication-defective adenovirus human strain 5 recombinant expressing the glycoprotein of the ERA strain of rabies virus was made as described in Xiang et al, Virol., 219:220-227 (1996) [Xiang III]. The recombinant, Adrab.gp [ATCC Accession No. VR-2554] was propagated and titrated on the E1-transfected 293 cell line [F. L. Graham et al, J. Gen. Virol, 36:59-72 (1977)]. For some of the experiments the virus was purified by CsCl gradient centrifigation as described in Y. Yang et al, Proc. Natl Acad. Sci.. USA, 90:9480-9484 (1993)].

95. 5. VRG

96. The vaccinia virus recombinant (VRG) recombinant which expresses the rabies virus glycoprotein of the ERA strain was propagated and titrated on HeLa cells as described in T. J. Wiktor et al, Proc. Natl. Acad. Sci., USA, 81:7194-7198 (1984) [Wiktor III].

97. D. Plasmid vector

98. The pSG5rab.gp vector which expresses the rabies virus glycoprotein of the ERA strain under the control of the simian virus (SV)-40 promoter was propagated in E. coli DH5a and purified using either kits from Promega or Qiagen according to the manufacturer's specifications. The vector was quantitated by agarose gel electrophoresis against a known standard. Details about construction of this plasmid have been described [see, Xiang II; S. R. Burger et al, J. Gen. Virol., 72:359-367 (1997), and Xiang et al, Virol., 199:132-140 (1994)].

### EXAMPLE 2

### Assay protocols

99. A. Enzynme Linked Immunosorbant Assay

100. Titers to rabies virus of sera obtained by retroorbital puncture were tested at serial dilution in duplicate or triplicate wells of microtiter plates coated with ERA-BPL virus and using an alkaline phosphatase goat anti-mouse immunoglobulin as second antibody [Xiang I]. Antibody isotypes were determined with a 1:200 dilution of serum using the Hybridoma Isotyping Kit (Calbiochem, San Diego, Calif.) according to the manufacturers specification with the modification of using plates coated with ERA-BPL virus [Wang 1997].

101. B. Cytokine Release Assay

102. Splenocytes from individual mice were co-cultured at 6×10⁶ nucleated cells without antigen or with 5 micrograms of ERA-BPL virus in 1.6 ml of culture medium supplemented with 10⁻⁶ M 2-mercaptoethanol and 2% FBS in 24 well Costar plates. Supernatants were harvested 24 hours later and co-cultured with 2×10³ HT-2 or CT4S cells in 200 microliters of culture medium supplemented with 10% FBS in microtiter plate wells. Proliferation of cells was determined 3 days later by 6 hour pulse with ³H-thymidine [H. Ertl et al., J. Virol., 63:2885-2892 (1990)].

103. C. Neutralization assay

104. Virus neutralizing antibody (VNA) titers were determined on BHK-21 cells infected with CVS-11 virus pretreated with serial dilution of heat-inactivated sera [Xiang I]. An NIH reference serum to rabies virus was tested at 10 international units (IU) for comparison. Data are expressed as IU derived by dividing the VNA titer of the experimental serum by that of the reference serum and multiplying the result by 10.

### EXAMPLE 3

### Immune Response to Neonatal Immunization with the Adrab.gp Recombinant

105. To summarize, the following studies show that neonatal vaccination with the Adrab.gp virus induced viral neutralizing antibodies (VNA) and T helper cells, resulting in protective immunity to rabies virus. The immune response was qualitatively indistinguishable from that seen in adult mice and could be achieved with different doses. More particularly, the VNA response could be elicited by different vaccine doses ranging from 10⁴ to 10⁸ pfu, and by different avenues of application including intranasal inoculation (data not shown), indicating that this vaccine, regardless of the dose or the route of vaccination, did not result in tolerance or the preferential activation of Th2 type responses as was described previously for another virus [Sarzotti, cited above].

106. A. Immunity to rabies virus in neonatal mice upon immunization with the Adrab.gp recombinant virus

107. Pups from naive C3H/He dams were vaccinated subcutaneously (s.c.) within 24 hours after birth with 10⁶ pfu of Adrab.gp virus (1st immunization), a vaccine dose that confers solid protection in adult mice. Control pups were inoculated with saline. Some of the pups of both groups were boosted at 2 month of age with 10⁶ pfu Adrab.gp virus given s.c. (2nd immunization). Pups were bled 2 weeks later and VNA titers were determined with CVS-11 virus on BHK-21 cells as described in H. Ertl et al., J. Virol, 63:2885-2892 (1990) (Ert1 I)].

108. As shown in Table 1, pups that received a single dose of the Adrab.gp virus at birth generated VNA titers comparable to those that were developed within 14 days by mice vaccinated at 2 month of age. A second immunization given to neonatally vaccinated pups at 2 month of age had a clear booster effect.

TABLE 1

| 1^{st} Immunization | 2^{nd} Immunization | VNA Titer |
|---|---|---|
| 10⁶ pfu Adrab.gp | None | 2278 |
| 10⁶ pfu Adrab.gp | 10⁶ pfu Adrab.gp | 6075 |
| None | 10⁶ pfu Adrab.gp | 2278 |
| None | None | <5 |

109. B. Assay to determine if varying the dose of antigen resulted in ‘tolerance’ or a switch towards a Th2 type response

110. Pups from naive C3H/He dams were inoculated within 48 hours after birth with a low (10⁴ pfu) or high (10⁸ pfu) dose of Adrab.gp virus or saline (none) administered s.c.. Serum VNA titers were tested 6 and 10 weeks later. As shown in Table 2, both vaccinated groups of mice developed high titers of antibodies to the rabies virus glycoprotein. The control (none) group is shown.

TABLE 2
Neonatal Immunization with a High and

| Vaccine | 6 weeks | 10 weeks |
|---|---|---|
| 10⁴ pfu Adrab.gp | 4252 | 28704 |
| 10⁸ pfu Adrab.gp | 3645 | 32805 |
| None | <5 | <5 |

111. C. Study to Determine if neonatal immunization resulted in a preferential Th1 or Th2 type immune response

112. The use of the Adrab.gp virus in adult mice provides an excellent protective immune response to rabies virus [Z. Q. Xiang et al, Virol,219:220-227 (1996) (Xiang III)]. Due to the deletion of the E1 gene the adenovirus recombinant fails to replicate (unless given in excessive doses) and carries thus a low risk of causing adverse reactions. The E1 deletion also affects synthesis of the E3 protein which is known to down-regulate expression of major histocompatibility antigens, thus inhibiting activation of CD8+T cells. Further, in adult mice, immunization with Adrab.gp virus elicits an antibody response that is predominated by IgG2a, the isotype reflecting a Th1 type response.

113. To test if neonatal immunization resulted in a preferential Th1 or Th2 type immune response, groups of C3H/He mice were immunized within 24 hours after birth with different doses, i.e., high (10⁸ pfu), intermediary (10⁶ pfu), or low (10⁴ pfu), of the Adrab.gp virus. The mice were bled 6 weeks later and tested for antibody isotypes (IgGI, IgG2a, IgG2b and IgG3) to rabies virus on plates coated with ERA-BPL virus. Serum from naive age-matched mice was used as a negative control; serum from mice immunized at 6-8 weeks of age with Adrab.gp virus 14 days previously was used as a positive control. All sera were used at a dilution of 1:200.

114. As shown in FIG. 1, the isotype profile of antibodies to rabies virus was similar in pups immunized as neonates with 10⁶ pfu of Adrab.gp to those derived from mice immunized as adults with the same dose (positive control). Pups vaccinated at birth with a high or low dose of the vaccine developed relatively more antibodies of the Th2 related isotypes (i.e., IgG1 and IgG2b). Nevertheless, in both groups the predominant response was that of the IgG2a isotype, indicating that neither dose had caused a switch towards a Th2 type response.

115. D. Development of a preferential Th1 type response upon neonatal immunization

116. The development of a preferential Th1 response upon neonatal immunization with the Adrab.gp vaccine was confirmed by testing splenocytes from pups for release of cytokines upon restimulation in vitro with inactivated rabies virus (see assay of Example 2B).

117. Mice were immunized at birth with 10⁶ pfu of Adrab.gp virus (6 mice) or saline (PBS, 4 mice). Mice were euthanized 6 weeks later and splenocytes from individual mice were co-cultured with medium or ERA-BPL virus. Supernatants of these cultures were tested for induction of proliferation of the HT-2 indicator cell line (Example 2B).

118. The results are shown in FIG. 2. Splenocytes from all of the immunized pups (6) secreted cytokines that induced proliferation of the HT2 cell line, an indicator cell line that is growth dependent on Interleukin (IL)-2 or 4. The culture supernatants failed to promote proliferation of CT4S cells (data not shown), an indicator cell line that responds exclusively to IL-4. None of the splenocytes from control pups secreted measurable levels of cytokines.

119. E. Protective immune response following neonatal immunization

120. To ensure that the immune response upon neonatal immunization resulted in protection, pups from naive C3H/He dams were immunized within 48 hours after birth with 1-2×10⁶ pfu of Adrab.gp and were challenged at 3 months of age with 10 LD₅₀ of the mouse virulent CVS-24 strain of rabies virus. All of the immunized mice (9 out of 9) survived, while all of the age-match control animals (15 out of 15) succumbed to the infection. Data is reported as Experiment I of Table 3.

121. In a subsequent experiment an additional group of neonatal pups was vaccinated s.c. with 1-2×10⁶ pfu of the Adrab.gp construct, and for comparison with the traditional inactivated rabies virus ERA-BPL vaccine given at 5-10 micrograms per pup s.c.. Mice were challenged with 10 LD₅₀ of CVS-24 virus given intramuscularly at 3 months of age. Pups vaccinated with adenoviral recombinant were again fully protected to challenge with virulent virus. None of the mice vaccinated as neonates with inactivated rabies virus survived the challenge with CVS-24 virus, as reported in Experiment II of Table 3.

TABLE 3

| | | | | |
|---|---|---|---|---|
| Dams | Pups | Vaccination | Challenge | Mortality |

| | | | | |
|---|---|---|---|---|
| PBS | Adrab.gp | At birth | 3 months | 0/9 |
| nothing | nothing | — | 3 months | 15/15 |

| | | | | |
|---|---|---|---|---|
| PBS | Adrab.gp | At birth | 3 months | 0/4 |
| PBS | ERA-BPL | At birth | 3 months | 6/6 |
| nothing | nothing | — | 3 months | 5/5 |

### EXAMPLE 4

### The Immune Response of Pups from Rabies Virus Immune Dams to the Adrab.gp Vaccine

122. In summary, the following studies demonstrate that vaccination of neonatal mice with the Adrab.gp construct showed initially a slight inhibition of the immune response in pups from rabies virus immune dams. The impairment of the antibody response to a single dose of the Adrab.gp vaccine was transient; several months after vaccination, pups from rabies virus immune dams showed higher antibody titers upon neonatal immunization with Adrab.gp virus compared to pups from naive dams. One potential explanation for this finding might be that maternal antibodies contribute to the disposition of rabies virus antigens in form of ICOSOMs on follicular dendritic cells, thus prolonging the B cell response [D. Gray, “Immunological Memory” in Immunogenicity, UCLA Symposium of Molecular and Cellular Biology (C. Janeway et al eds), Alan R. Liss, NY, pp.219-228 (1990)].

123. A. Effect of maternally transferred antibodies to rabies virus oil immune response to the Adrab.gp vaccine.

124. Adult female mice were inoculated 2-3 times in a fourteen day interval with 2-10 micrograms of ERA-BPL virus. Mice were bled 7-10 days after the booster immunization to determine antibody titers. They were then co-housed with naive syngeneic male mice.

125. The offspring of the female C3H/He mice immunized as described above were vaccinated subcutaneously at 10 weeks of age with Adrab.gp virus (10⁶ pfu) or inactivated ERA-BPL virus (10 μg). Control dams and offspring received no immunizations. Mice were bled by retro-orbital puncture. Serum was prepared and stored at −20° C. Serum VNA titers were tested 2, 4 and 6 weeks post-vaccination.

126. As shown in Table 4, overall, the rabies virus specific VNA response to the Adrab.gp construct was clearly superior to the response elicited to the inactivated rabies virus. Furthermore, the antibody response to the rabies virus glycoprotein was strongly inhibited in pups from rabies virus immune dams upon vaccination with ERA-BPL virus. Titers were low 2 and 4 weeks after vaccination and then declined rapidly to levels below detectability by 6 weeks after vaccination. In contrast, the immune response to the Adrab.gp vaccine was comparable in magnitude in pups from naive and rabies virus immune dams thus demonstrating that maternal immunity to rabies virus did not affect the B cell response to the rabies virus glycoprotein presented by an adenoviral recombinant.

TABLE 4
VNA response to the Adrab.gp vaccine

VNA Titers

| Dams | Pups | 2 | 4 | 6 |
|---|---|---|---|---|
| PBS | ERA-BPL | 45 | 80 | 30 |
| ERA-BPL | ERA-BPL | 15 | 25 | <5 |
| PBS | Adrab.gp | 2300 | 6075 | 2300 |
| ERA-BPL | Adrab.gp | 2600 | 6075 | 2300 |
| PBS | nothing | <5 | <5 | <5 |

127. B. Antibody response to rabies virus in pups from rabies virus-immune dams

128. The following experiment was performed to determine if the lack of an VNA response upon vaccination of pups from immune dams with inactivated rabies virus was compensated for by the development of non-neutralizing antibodies to other antigens present in the inactivated rabies virus vaccine.

129. Mice from naive or rabies virus immune dams were vaccinated at 10 weeks of age with 5 micrograms of whole inactivated ERA-BPL rabies virus or 1-2×10⁶ pfu of Adrab.gp virus. Mice were bled 1 month later and antibody titers were determined by ELISA, performed as described in Example 2A.

130. The antibody response to rabies virus was completely inhibited in pups from rabies virus immune dams vaccinated with ERA-BPL virus. The same group of pups vaccinated with the Adrab.gp construct showed an excellent immune response that in this experiment was even slightly superior to that seen in pups from naive dams. Data were thus consistent with those obtained by the neutralization assay.

131. C. Protective Immune Response in Vaccinated Pups

132. In a separate experiment, pups from naive or ERA-BPL rabies virus immune dams were vaccinated at 6 weeks of age with 5-10 micrograms of ERA-BPL virus or 1-2×10⁶ pfu of the Adrab.gp construct. Pups as wells as age-matched naive C3H-He mice were challenged at 5 months of age with CVS-24 virus (Experiment III). In another experiment, pups from naive or ERA-BPL virus immune dams were vaccinated within 48 hours after birth with 4×10⁴ pfu of Adrab.gp virus. Mice were challenged at 9 months of age with CVS-24 virus. In this experiment, 7 months old naive C3H/He mice were used as controls.

133. Beginning 7 days following challenge, mice were observed daily for symptoms indicative of a rabies virus infection. Mice that developed complete bilateral hindleg paralysis, a sign for the terminal stage of rabies, were euthanized for humanitarian reasons. Upon challenge unvaccinated mice died within 8-12 days. Surviving mice were kept and observed for an additional 2-3 weeks to ensure that they survived the infection.

134. As shown in Table 5 at Experiments I and II, all of the Adrab.gp vaccinated mice were protected while all of the mice immunized with inactivated rabies virus vaccine succumbed to the infection. In Table 5, mortality reflects the number of dead mice/total number of mice in the experiment.

TABLE 5

| | | | | |
|---|---|---|---|---|
| Dams | Pups | Vaccination | Challenge | Mortality |

| | | | | |
|---|---|---|---|---|
| ERA/BPL | ERA/BPL | 6 weeks | 5 months | 8/9 |
| ERA-BPL | Adrab.gp | 6 weeks | 5 months | 0/9 |
| nothing | nothing | — | 5 months | 6/6 |

| | | | | |
|---|---|---|---|---|
| ERA-BPL | Adrab.gp* | At birth | 9 months | 0/9 |
| PBS | Adrab.gp | At birth | 9 months | 0/6 |
| nothing | nothing | — | 7 months | 5/5 |

135. Accordingly pups vaccinated at birth with ERA-BPL virus were not protected to a challenge with live rabies virus given at 3 months of age (See Experiments I and II of Table 5).

136. D. Use of Recombinant Viral Vaccines to Overcome Maternal Inhibition

137. To test if recombinant viral vaccines in general could overcome maternal inhibition to rabies virus in pups from ERA-BPL virus immune dams, a similar experiment was conducted as follows. Pups from naive (PBS) or ERA-BPL immune C3H/He dams were vaccinated at about 2 months of age with 10⁶ pfu of the recombinant vaccinia vaccine carrying the rabies glycoprotein (VRG) (see Example IC5). In this experiment the vaccine was given i.p. rather than s.c., the route of administration chosen for the Adrab.gp virus. Mice were bled 2 weeks later and VNA titers were determined.

138. As shown in Table 6, the VNA response to rabies virus upon vaccination with the VRG construct was strongly reduced in pups from ERA-BPL virus immune dams, suggesting that at least this recombinant vaccine did not overcome maternal interference. Varying the route of administration had little effect on the vaccine efficacy of the VRG construct in pups from rabies virus immune dams (data not shown).

TABLE 6

| Dams | Pups | VNA Titer |
|---|---|---|
| PBS | VRG | 27337 |
| ERA-BPL | VRG | 150 |
| PBS | Nothing | <5 |

139. In summary, the VRG recombinant elicited a markedly decreased B cell response in presence of maternal antibodies. The VRG virus is cytopathic, i.e., kills infected cells within hours causing release of new infectious virus particles as well as fragments of antigen. The B cell response to the VRG vaccine was largely dependent on antigen released by cells dying as a consequence of the viral infection, and such antigen was neutralized or retargeted to inappropriate APCs by maternal antibodies.

140. E. Neonatal Immune Response in the Presence of Maternal Immunity

141. To test if the neonatal immune response to the Adrab.gp vaccine was inhibited in the presence of maternal immunity to rabies virus, pups from rabies virus immune (ERA-BPL vaccinated) and naive (PBS) C3H/He dams were immunized within 48 hours after birth with about 4×10⁶ pfu of Adrab.gp virus. ERA-BPL virus was not included in this experiment. Serum VNA titers were determined 4, 8 and 12 weeks later.

142. As shown in Table 7, pups from naive and ERA-BPL immune dams developed VNAs to rabies virus. The response was, at these time points, slightly superior in pups from naive dams, indicating that the high levels of maternally transferred antibody present at birth and in suckling mice for the initial postnatal phase might have slightly inhibited the antibody response to the rabies virus glycoprotein.

TABLE 7
Neonatal VNA response to the Adrab.gp vaccine in pups

VNA Titers

| Dams | Pups | 4 | 8 | 12 |
|---|---|---|---|---|
| PBS | Adrab.gp | 1822 | 3645 | 1215 |
| ERA-BPL | Adrab.gp | 675 | 1012 | 607 |

143. F. Effect of maternally transferred antibodies to the rabies virus glycoprotein of the long-term immune response

144. The same groups of pups, i.e., mice from naive or ERA-BPL immune dams, were immunized at birth with about 4×10⁶ pfu of Adrab.gp. Mice were bled at 6 and 8 months of age and serum antibody titers to rabies virus were tested by an ELISA. This method more readily detects minor differences in titer. Antibody titers in both groups of mice were high in magnitude 6 months after immunization, indicating that the impairment of the VNA response seen shortly after vaccination was transient. At 8 months of age, the antibody titers started to decline in pups from naive dams, while those of pups from immune dams remained high. See FIGS. 10A, 10B and 10C.

145. Pups from naive or ERA-BPL virus immune dams were vaccinated within 48 hours after birth with 4×10⁴ pfu Adrab.gp virus. The mice were challenged at 9 months of age with 10 LD₅₀ of CVS-24 virus and then bled 3 weeks after challenge. Seven month old naive C3H/He mice were used as controls. Antibody titers to rabies virus were tested by an ELISA. Mice from rabies virus immune dams immunized at birth with the Adrab.gp construct showed again slightly higher antibody titers compared to pups from naive dams. In the challenge experiment, both neonatally Adrab.gp vaccinated pups from naive or rabies virus immune dams survived the infection with the CVS-24 strain of rabies virus, which killed all of the control mice. See the results in Table 8 below.

TABLE 8

| Dams | Pups | Vaccination | Challenge | Mortality |
|---|---|---|---|---|
| ERA-BPL | Adrab.gp | 0-48 hours | 9 months | 0/9 |
| PBS | Adrab.gp | 0-48 hours | 9 months | 0/6 |
| None | None | | 7 months | 5/5 |

146. In summary, the immune response to the E1-deleted, replication defective adenoviral recombinant which expresses the glycoprotein of rabies virus under the control of the potent CMV promoter, was not impaired by the existing maternal immunity. The adenoviral recombinant due to the E1 deletion is noncytolytic thus readily establishing persistent infection in vitro as well as in vivo. The adenoviral recombinant presumably initiates a B cell response via surface expressed glycoprotein which might be less amenable to neutralization or retargeting.

### EXAMPLE 5

### The Effect of Maternally Transferred Immunity on the Efficacy of a Genetic Vaccine in Adult Mice

147. Genetic vaccines do not express protein antigens until de novo synthesis is initiated in transfected cells. At the initial stage upon inoculation, genetic vaccines are neither susceptible to neutralization nor re-targeting by antibodies. Thus, such vaccine compositions are expected to provide an avenue to overcome maternal interference. In a manner similar to that of the El-deleted adenoviral recombinant, genetic vaccines do not lead to the demise of transfected cells and induction of B cell responses by nonsecreted antigens, such as the rabies virus glycoprotein that is firmly anchored into the cell membrane and is assumed to rely on membrane expressed protein.

148. To test the effect of either maternally transferred immunity or passively administered antibodies on genetic immunization of mice, a series of experiments was conducted in either young adult or neonatal mice. The following results show that in adult mice passively acquired immunity, either by maternal transfer or upon inoculation of a hyperimmune serum, strongly reduces the B cell response to the genetic vaccine. Surprisingly, this effect was much less pronounced upon immunization of neonates.

149. A plasmid vector, termed pSG5rab.gp (Example 1D), expressing the glycoprotein of rabies virus was tested for induction of an antibody response in the presence of maternally transferred immunity or passively transferred antibodies to rabies virus in young adult or neonatal mice. Six week old mice born to rabies virus glycoprotein immune dams developed an impaired antibody response to genetic immunization as had been previously observed upon vaccination with an inactivated viral vaccine. Similarly, mice passively immunized with a hyperimmune serum showed an inhibited B cell response upon vaccination with the pSG5rab.gp vector resulting in both cases in vaccine failures upon challenge with a virulent strain of rabies virus. In contrast the immune response of mice vaccinated as neonates in the presence of maternal immunity or upon passive immunization to rabies virus with the pSG5rab.gp construct was only marginally affected.

150. A. Adult female C3H/He mice were vaccinated twice with 5 μg of EPA-BPL inactivated rabies virus vaccine given i.m. prior to mating. Control mice were inoculated with saline. Both groups of females were mated 2 weeks after the second immunization with syngeneic males. Male and female pups were vaccinated at 6 weeks of age, when maternal antibodies had declined, with either 5 μg of ERA-BPL virus given s.c. or 50 μg of the pSGSrab.gp vector given i.m. Mice were bled 6 weeks later and serum antibody titers were tested by an ELISA (Example 2A) on plates coated with inactivated rabies virus.

151. As shown in FIG. 3, pups from rabies virus-immune dams developed upon immunization with either vaccine reduced antibody titers in comparison to pups from sham-vaccinated dams, indicating that the immune response to the genetic vaccines was as affected by maternal transferred immunity as the viral vaccine. The rabies virus vaccine induces antibodies to a number of viral proteins most notably the nucleoprotein in addition to the viral glycoprotein. The pSG5rab.gp vaccine on the other hand stimulates a monospecific response to the viral glycoprotein, the sole target antigen of rabies virus neutralizing antibodies (VNA) the main immune correlate of protection.

152. B. The same batch of sera tested by ELISA as described above was next tested for VNA titers to rabies (Example 2C). The results of the biological assay confirmed those of the ELISA. Sera of pups from rabies virus immune dams had reduced antibody titers upon immunization with either of the two vaccines compared to sera of control pups. Nevertheless, VNA titers were higher in either group of pSG5rab.gp vaccinated mice than mice immunized with inactivated rabies virus which gave raise to measurable albeit low titers in pups from naive dams but not in pups from immune dams (FIG. 4A).

153. C. Pups immunized with the pSG5rab.gp were next, i.e., 8 weeks after immunization, challenged with 10 mean lethal doses (LD₅₀) given i.m. of the mouse-adopted virulent CVS-24 virus strain of rabies virus which is antigenically closely related to the ERA strain. Mice were observed daily starting 7 days later. Mice were euthanized once they developed bilateral hindleg paralysis, a definite symptom of a terminal rabies virus infection. Mice that survived the infection were observed for an additional 14 days. Mice were subsequently bled to assess the booster effect of the challenge.

154. Protection as expected paralleled VNA titers. All of the pSG5rab.gp vaccinated pups from naive dams remained symptom-free, while 20% of DNA vaccinated pups from immune dams succumbed to the infection (FIG. 4B). VNA titers in surviving pSG5rab.gp vaccinated mice were tested 2 weeks after challenge, and demonstrated that injection of live virus had a clear booster effect, indicating that the vaccine had not induced sterilizing immunity in either group. Again postchallenge titers were higher in pups born to naive dams than in pups from rabies virus immune dams (FIG. 4A).

155. D. To further ascertain that maternal immunity to the rabies virus glycoprotein impaired the offspring's B cell response to the pSG5rab.gp vaccine, this experiment was conducted. Female C3H/He mice were immunized twice prior to mating with either 10⁶ pfu of Adrab.gp virus or with PBS. Pups from the Adrab.gp vaccinated dams were themselves vaccinated at 6 weeks of age with 5μg of ERA-BPL virus or with 50 μg of pSG5rab.gp vector. Pups from the PBS (sham) vaccinated dams were themselves vaccinated at 6 weeks of age with 5μg of ERA-BPL virus or with 50 μg of pSG5rab.gp vector. The mice were bled 6 weeks later and serum antibody titers were determined by an ELISA using age-matched normal mouse sera for comparison and the neutralization assay (Example 2A and 2C). See FIG. 5.

156. Adrab.gp virus, like the genetic vaccine, induces a monospecific response to the glycoprotein of rabies virus, as well as responses to the adenoviral antigens. Mice born to Adrab.gp immune dams immunized with the either vaccine showed a strongly reduced antibody response which in pups vaccinated with the vector was below the level of detectability. The neutralization assay confirmed these results. Pups born to immune dams vaccinated with either construct developed VNA titers of 1:15 which are at the lowest level of reliable detectability while pups from naive dams vaccinated with the viral vaccine or the vector had VNA titers of 1:135 and 1:405 respectively.

### EXAMPLE 6

### The Effect of Passive Immunization on the Immune Response to a DNA Vaccine

157. To test if antibodies directly affect the efficacy of the genetic vaccine, the immune response to the pSG5rab.gp vector in mice passively immunized to rabies virus was tested. Groups of adult C3H/He mice were inoculated i.p. with 200 μl of a syngeneic hyperimmune serum to ERA-BPL virus containing 10 IU of VNA to rabies virus. Control mice were inoculated with an equivalent dose of normal C3H/He mouse serum. Resulting serum VNA titers were determined the following day (Example 2C).

158. Mice inoculated with the hyperimmune serum had 3 IU of circulating VNA, control mice were negative. Four days following passive immunization, mice were vaccinated either with 50 μg of the pSG5rab.gp vector given i.m. or with 10 μg of ERA-BPL virus given s.c. Serum antibody titers to rabies virus were tested 6 weeks later by an ELISA (Example 2A).

159. As shown in FIG. 6A, mice inoculated with serum to rabies virus developed an impaired antibody response upon vaccination with the inactivated viral vaccine. Inhibition was also seen upon genetic vaccination, confirming the results obtained in mice born to rabies virus immune dams.

160. Mice were later challenged with 10 LD₅₀ of CVS-24 virus. As shown in FIG. 6B, all of the passively immunized mice vaccinated with the pSG5rab.gp construct succumbed to the infection while genetically-vaccinated control animals were completely protected.

### EXAMPLE 7

### The Effect of Maternal Immunity on the B-Cell Response of Neonatal Mice to Genetic Immunization

161. The effect of maternal immunity on the B cell response upon genetic vaccination of neonatal mice was tested as follows. Pups born to C3H/He dams vaccinated with ERA-BPL virus or a sham vaccine were inoculated within 48 hours after birth with 50 μg of the pSG5rab.gp vector given s.c.. ERA-BPL virus that fails to induce a measurable immune response in neonatal mice was not included in this set of experiments. Mice were bled 1, 2, 4, 6 and 8 months later, and serum antibody titers were determined by ELISA (Example 2A) using a normal mouse serum from 8-10 week old mice for comparison.

162. The results are reported in FIGS. 7A through 7D. At the earliest time point tested, i.e., 1 month after immunization, antibody titers were a great deal higher in pups born to rabies virus immune dams, which is most likely a reflection of residual maternal antibodies. These antibodies decreased but were still detectable 2 months after vaccination. Later on at 4, 6 and 8 months of age antibody titers of pups from immune pups eventually declined below those of pups from naive pups. Nevertheless, the differences in titers were marginal compared to that seen upon immunization of 6 week old pups from naive or rabies virus immune dams or upon passive transfer of antibodies prior to genetic immunization of adult mice.

163. To ensure that the slight difference observed in pups from immune dams, shown in 3 separate experiments, was not within the limits of natural variability (which is rather high upon genetic immunization), mice were boosted at 10 weeks of age with a low dose (i. e., 10⁴ pfu) of an E1-deleted adenoviral recombinant. As shown in FIG. 8, both groups of mice rapidly developed an anamnestic B cell response to the rabies virus antigen that was clearly superior in mice born to naive dams.

### EXAMPLE 8

### The Effect of Passive Immunization on the Immune Response of Neonatal Mice to Genetic Immunization

164. To further evaluate the effect of pre-existing antibodies on the immune response of mice inoculated as neonates with the pSG5rab.gp vaccine, groups of C3H/He mice were injected within 48 hours after birth with 10 IU of a hyperimmune serum to rabies virus or an equivalent dose of a normal mouse serum both derived from syngeneic donors. Mice were then vaccinated with 50 μg of the pSG5rab.gp vaccine. Antibody titers to rabies virus were tested 3 and 6 months later by an ELISA.

165. As shown in FIGS. 9A and 9B, at both time points titers from pups vaccinated in the presence of antibodies to rabies virus or a normal serum preparation were indistinguishable. These results are in stark contrast to the results obtained upon genetic vaccination of passively immunized adult mice.

### EXAMPLE 9

### The Effect of Maternal Immunity on the Isotype Profile of the Antibody Response to Genetic Vaccination

166. The isotype profile of antibodies to rabies virus from mice immunized as neonates with the pSG5rab.gp vaccine was determined to establish if the presence of maternally transferred immunity had shifted the type of the response. Sera harvested from pups born to naive or rabies virus-immune dams vaccinated as neonates with the pSG5rab.gp construct were tested 5 and 7 months later for the distribution of isotypes of antibodies on ERA-BPL coated plates by an ELISA.

167. As shown in FIG. 10, both groups of mice had the same antibody isotype profile to rabies virus with IgG2a being clearly predominant thus being indicative of a Th 1 type response.

168. In summary, young adult mice born to rabies virus immune dams or to Adrab.gp immune dams consistently developed impaired B cell responses to genetic immunization compared to control mice born to naive dams. Passive immunization had the same effect suggesting that the pathway of B cell activation upon genetic immunization with the rabies virus glycoprotein expressing vector is susceptible to interference by passively transferred antibodies. Surprisingly this interference was much less pronounced upon genetic vaccination of neonates. Accordingly neonates vaccinated with the pSG5rab.gp vector developed antibody titers to rabies virus that were identical to those of control mice inoculated with a normal mouse serum instead. It is unclear why young adult mice and neonatal mice responded differently to the genetic vaccine given in presence of passively acquired immunity. However, data presented here clearly indicates such a qualitative difference.

169. Numerous modifications and variations of the present invention are included in the above-identified specification and are expected to be obvious to one of skill in the art. Such modifications and alterations to the compositions and processes of the present invention are believed to be encompassed in the scope of the claims appended hereto.

What is claimed is:

1. A method for overcoming maternal inhibition to a vaccine and inducing a protective immune response in a mammalian infant of a selected species against infection comprising the step of:
administering to said infant at an age of under 1 year a composition comprising a suitable dose of a transcribable polynucleotide sequence comprising a sequence encoding an antigen of a pathogenic organism.

2. The method according to claim 1 wherein said polynucleotide sequence is under the regulatory control of a promoter, wherein neither the polynucleotide sequence nor the antigen causes a pathogenic infection in said infant.

3. The method according to claim 1 wherein said selected species is human.

4. The method according to claim 1 wherein said polynucleotide sequence comprises a recombinant virus.

5. The method according to claim 4 where in said virus is a replication-defective virus.

6. The method according to claim 4 wherein said virus is selected from the group consisting of adenovirus, poxvirus, and retrovirus.

7. The method according to claim 1 wherein said polynucleotide sequence comprises a DNA vaccine.

8. The method according to claim 1 wherein said organism is selected from the group consisting of rabies virus, respiratory syncytial virus, rotavirus, human immunodeficiency virus, and measles virus.

9. The method according to claim 8 wherein said organism is rabies virus and said polynucleotide sequence is present in a recombinant adenovirus vector Adrab.gp [ATCC Accession No. VR-2554].

10. The method according to claim 1 wherein said polynucleotide sequence is administered in a suitable pharmaceutical carrier.

11. The method according to claim 4 wherein the viral dose is about 10⁴ to about 10⁷ pfu recombinant virus.

12. The method according to claim 8 wherein said wherein said organism is rabies virus and said polynucleotide sequence is present in a plasmid vector, pSG5rab.gp.

13. The method according to claim 7 wherein the dose is between about 0.5 μg to about 5 mg plasmid vector.

14. The method according to claim 1 which is a veterinary method and said infant is a newborn animal.

15. The method according to claim 14 wherein said mammalian species is selected from the group consisting of a domestic animal or livestock.

16. The method according to claim 14 wherein said polynucleotide sequence causes a pathogenic infection in humans.

17. Use of a suitable dose of a transcribable polynucleotide sequence comprising a sequence encoding an antigen of a pathogenic organism in the preparation of a medicament for overcoming maternal inhibition to a vaccine and inducing a protective immune response in a mammalian infant of less than one year of age against infection.

18. Use according to claim 17, wherein neither the polynucleotide sequence nor the antigen causes a pathogenic infection in said infant.

19. Use according to claim 17 wherein said selected species is human.

20. Use according to claim 17 wherein said polynucleotide sequence comprises a recombinant virus.

21. Use according to claim 20 wherein said virus is a replication-defective virus.

22. Use according to claim 20 wherein said virus is selected from the group consisting of adenovirus, poxvirus, and retrovirus.

23. Use according to claim 17 wherein said polynucleotide sequence comprises a DNA vaccine.

24. Use according to claim 17 wherein said organism is selected from the group consisting of rabies virus, respiratory syncytial virus, rotavirus, human immunodeficiency virus, and measles virus.

25. Use according to claim 24 wherein said organism is rabies virus and said polynucleotide sequence is a recombinant adenovirus vector Adrab.gp [ATCC Accession No. VR-2554].

26. Use according to claim 17 wherein said polynucleotide sequence is administered in a suitable pharmaceutical carrier.

27. Use according to claim 20 wherein said suitable dose is about 10⁴ to about 10⁷ pfu recombinant virus.

28. Use according to claim 24 wherein said wherein said organism is rabies virus and said polynucleotide sequence is a plasmid vector, pSG5rab.gp.

29. Use according to claim 28 wherein said suitable dose is between about 0.5 μg to about 5 mg plasmid vector.

30. Use according to claim 17 which is a veterinary method and said infant is a newborn animal.

31. Use according to claim 30 wherein said mammalian species is selected from the group consisting of a domestic animal or livestock.

32. Use according to claim 30 wherein said polynucleotide sequence causes a pathogenic infection in humans.
